(19) United States
(12) Patent Application Publication
(10) Pub. No.: US 2004/0013698 A1
(43) Pub. Date: Jan. 22, 2004

(54) Method for preparing topical preparations containing skin protective agents with enhanced barrier properties

(76) Inventors: Duncan T Aust, Carson City, NV (US); James A Hayward, Stony Brook, NY (US); James M Wilmott, Shoreham, NY (US)

Correspondence Address: Robert C Sullivan, Darby & Darby, Post Office Box 5257, New York, NY, 10150-5257, US

(21) Appl. No.: 10/332,394

(22) PCT Filed: Jul. 9, 2001
(86) PCT No.: PCT/US01/21578

(51) Int. Cl. A61K 7/00

(52) U.S. Cl. 424/401; 514/54

(57) ABSTRACT

Disclosed are methods for preparing topical compositions with enhanced barrier properties comprising dispersions of hydrophobic skin protective agents.

### FIELD OF THE INVENTION

The present invention is a method for making a composition comprising an oil-in-water dispersion composed of one or more hydrophobic skin protective agents, a hydrophilic Theological modifying agent, and an aqueous phase.

### BACKGROUND OF THE INVENTION

Elements of the invention are described in the ensuing text. This description is provided as information to help define the extent of the invention and is non-limiting in nature.

Compositions containing skin protective agents such as petrolatum or silicone are commonly used in formulations for topically applied pharmaceutical or cosmetic products. Petrolatum and silicone are particularly desirable for use in formulations because of their relatively high water resistance, and their ability to form a barrier on the skin for skin protection.

These ingredients typically are not used without some modification, because in their unmodified state they are aesthetically unappealing and diminish the consumer appeal of products into which they are incorporated.

In an effort to effectively incorporate these ingredients into topical preparations, skin protective agents are typically either solubilized in a suitable hydrophobic fluid, or are incorporated into water-in-oil (W/O) or oil-in-water (O/W) emulsions. However, emulsions present problems both in processing and in the selection of ingredients used to form the emulsions.

Emulsions are prepared by adding emulsifiers to the hydrophobic and hydrophilic phases of a composition. Emulsifiers are a class of surface active ingredients, commonly called surfactants, which reduce the surface tension of water and the interfacial tension between the hydrophobic and hydrophilic phases. However, the current methods for preparing O/W emulsions form internal phase particles which are typically 1 to 5 microns or greater in size.

O/W emulsions are typically prepared by first separately creating the hydrophobic and hydrophilic phases. A hydrophilic phase, containing water and water compatible emulsifiers and components, is prepared and heated with mixing to a temperature in excess of 70° C. A hydrophobic phase containing the non-polar ingredients such as oils, and oil compatible emulsifiers and components, is prepared and also heated with mixing to a temperature in excess of 70° C. until a homogeneous preparation results. The hydrophobic phase is added to the hydrophilic phase and mixed with suitable agitation to intimately intermingle the phases. The total composition is then cooled to remove the excess heat until the composition reaches ambient temperature.

The particle size and stability of the hydrophobic phase depends upon the emulsifiers used. The selection of emulsifiers is governed by the hydrophobic-hydrophilic balance (HLB). Failure to select a suitable combination of emulsifier, hydrophobic and hydrophilic materials will result in a magnified incompatibility between the phases resulting in increased particle size and physical instability. Further, emulsifiers tend to create large micellularized structures of the hydrophobic phase in the hydrophilic phase.

The particle size of an emulsion is also dependent on the processing conditions such as the rate and degree of heating and cooling, and on the type and duration of shear used during mixing. If any one of the processing conditions are not precisely duplicated, or if the ingredients used in the composition are not of the same nature and quality in each batch prepared, reproducibility of the properties and performance of the compositions is compromised. For example, if the particle size of the emulsion changes then the nature of the film left on the skin after application will also change. This compromises the beneficial properties of materials such as petrolatum and silicone which are used to enhance the barrier properties of the skin. When applied to the skin, emulsifiers can alter the structure of the indigenous lipids present between and within cell membranes that constitutes the natural barrier properties of the skin. When the natural barrier is compromised, channels for passage of materials both into and out of the skin are opened. The emulsifier, as well as other substances from the composition, such as preservatives, fragrances, actives or chelating agents, can penetrate into and irritate the skin. In addition, greater amounts of water will be released from the lower layers of the skin to the skin surface and to the surrounding atmosphere. As a consequence, adverse skin conditions including erythema, edema, allergic responses such as rashes and itching, dryness, fissures, roughness, scaliness and irritant contact dermatitis develop.

The type and quantity of emulsifier used also dictates the type and nature of the film that is produced on the skin's surface. As the topically applied preparation dries, the volatile components, principally water, are either absorbed by the skin or are evaporated into the surrounding atmosphere. The residual hydrophobic materials and non-volatile hydrophilic materials are either absorbed into the skin or left behind at the surface to create a film. These materials often compromise the beneficial properties of materials such as petrolatum and silicone which are used to enhance the barrier properties of the skin.

Accordingly, it is desirable to produce a stable topical formulation comprising a surfactant-free oil-in-water dispersion of a skin protective agent such as petrolatum or silicone, having enhanced barrier properties for the skin or hair, more predictable film-forming properties, and aesthetically pleasing tactile properties.

### SUMMARY OF THE INVENTION

The present invention provides method for preparing a topical composition with enhanced barrier properties comprising mixing an oil-in-water dispersion comprising one or more hydrophobic skin protective agents, and a base composition comprising a hydrophilic Theological modifying agent and an aqueous phase.

In a preferred embodiment, the oil-in-water dispersion comprising one or more hydrophobic skin protective agents is prepared by high pressure/high shear mixing conditions to form a stable oil-in-water dispersion having a particle size of from about 50 to 1000 nm.

The base composition is prepared by mixing an aqueous phase, comprising water and water compatible components, and one or more hydrophilic rheological modifying agents. The dispersion and base composition are them mixed to form the topical composition.

According to the method of the invention, the dispersions of the present invention are produced by mixing from about 10 to 60 wt %, and preferably from about 25 to 55 wt % of one or more hydrophobic skin protective agents, with from about 40 to 90 wt % aqueous phase, and processing the mixture under high pressure, high shear, or high pressure/high shear conditions until a particle size of from about 50 to 1000 nm, preferably from about 100 to 800 nm is obtained.

The method of the invention produces a dispersion having a specific gravity of from about 0.8 to 1.1, preferably from about 0.85 to 0.95.

The method of the invention produces a dispersion having a viscosity of less than about 10000 cps.

The pH of the dispersion is between about 4.5 and 7.5, and preferably between about 5.0 and 7.0.

In one embodiment of the method of the invention, the hydrophobic skin protective agent is a petrolatum or silicone or mixtures thereof. The petrolatum, silicone or mixtures thereof are present in an amount of from about 10 to 60 wt %; and preferably from about 25 to 55 wt % based on total weight of the dispersion. The aqueous phase of the dispersion is present in an amount of from about 40 to 90 wt % based on total weight of the dispersion.

The compositions of the present invention are produced by mixing from about 1 to 95 wt % of the dispersion comprising a hydrophobic skin protective agent such as petrolatum, silicone or combination thereof, with a base composition comprising from about 0.01 to 10 wt % of one or more hydrophilic Theological modifying agents and from about 5 to about 99 wt % water, based on total weight of the final composition.

The present invention also provides compositions comprising dispersions prepared by the method of the invention and a base composition as described above.

### BRIEF DESCRIPTION OF THE FIGURE

FIG. 1 shows the TEWL data obtained in Example 9.

### DETAILED DESCRIPTION OF THE INVENTION

It has been surprisingly found that topical compositions of the present invention have enhanced moisture retention properties and provide protection to the skin from external irritants better than prior art compositions.

All patents, applications, test methods and publications referenced in this specification are hereby incorporated by reference in their entirety. In case of Conflict, the present description, including definitions, will prevail.

As used herein the term “surface-active” or “surface-active agent” refers to a substance capable of reducing the surface tension of a liquid in which it is dissolved and which modifies the interfacial tension between the hydrophilic and hydrophobic phases.

A “non-surface active agent” is a substance which does not effectively reduce the surface tension of a liquid in which it is dissolved or dispersed.

As used herein, the term “surfactant” refers to a surface-active substance.

As used herein, the term “surfactant-free dispersion” refers to a stable dispersion that is produced without the use of surface-active ingredients or surfactants.

The term “dispersion” or “oil-in-water dispersion” are used interchangeably herein, and refer to the suspension of an oil (or nonpolar substance) in a polar (for example, aqueous) composition.

A preferred method of processing the oil or hydrophobic phase with water is through the use of high pressure, high shear mixing or high pressure/high shear mixing. The high pressure, high shear mixing or high pressure/high shear mixing may be performed using suitable equipment including homogenizers, microfluidizers and ultrasonic mixers. The preferred pressure for preparing the dispersion is between about 11,000 psi to about 25,000 psi with a desired shear that creates an average particle size of between about 50 nm to about 1000 nm.

The dispersions of the current invention can be either non-ionic or cationic in nature. Non-ionic dispersions are formed by mixing the hydrophobic skin protecting agent with a non-surface active phospholipid and water, homogenizing the mixture and subjecting the homogenized mixture to high pressure/high shear mixing at pressures of from about 9,000 to about 25,000 psi. Preferred phospholipids include Phospholipon 80, 80H (American Lecithin Co., Oxford, Conn.), Basis LP20H (Ikeda Corp., Japan), and Catemol, a synthetic lipid-like compound (Phoenix Chemicals Inc., Somerville, N.J.).

Cationic dispersions are formed by mixing the hydrophobic skin protecting agent with a combination of a C₁₀-C₅₀ amido amine with a C₁₀ to C₅₀ carboxyllic acid and water, then homogenizing the mixture to improve its homogeneity and subjecting the mixture to high pressure/high shear mixing at pressures of about 9,000 to about 25,000 psi. A preferred cationic material is the combination of stearic acid and stearamidopropyldimethylamine (Phoenix Chemical, Inc., Somerville N.J.).

Alternatively, homogenization may be replaced by heating. Heating is preferred for viscous hydrophobic skin protecting agents such as petrolatum. Heating reduces the viscosity of the material, thereby facilitating the subsequent high pressure/high shear mixing.

Hydrophobic skin protective agents are included in the composition of the current invention. These materials are designed to improve the barrier properties of the skin as manifested by greater water retention or enhanced protection from external irritants or sensitizers. Suitable skin protective agents include petrolatum, water-insoluble silicones and fluorocarbons.

Suitable water-insoluble silicone materials include, but are not limited to, cyclomethicone, polyalkylsiloxanes, polyarylsiloxanes, polyalkylarylsiloxanes, polysiloxane gums and polyethersiloxane copolymers. Examples of suitable silicone materials are disclosed in U.S. Pat. Nos. 4,788,006; 4,341,799; 4,152,416; 3,964,500; 3,208,911; 4,364,837 and 4,465,619, all of which are incorporated herein by reference.

Exemplary silicone and silicone derivatives include branched or linear cyclical silicone or silicone derivatives, cyclomethicone, dimethicone polysiloxane, dimethiconol, polysiloxanes, polysiloxane copolymers, polyalkyl aryl silanes, polyaryl siloxanes, polyalkyl siloxanes, polyalkyl aryl silanes, polysiloxane copolymers. Preferred examples of silicone solvents or co-solvents include: low viscosity dimethicone, phenyl trimethicone (Dow Corning) and silicone fluid DC 345 (Dow Corning).

Suitable fluorocarbons include, but are not limited to, linear, saturated, unsaturated, cyclic or branched compounds containing one or more mono-, di-, or tri-fluoro substituted methylene units. An exemplary fluorocarbon is perfluoropolymethylisopropyl ether sold under the trade name of Fomblin™ HC/25 by Brooks Industries of South Plainfield, N.J.

Base Composition

Hydrophilic Theological modifying agents within the scope of the invention include any substance which increases or decreases the viscosity of the composition of the invention. Suitable Theological modifying agents include, but are not limited to, phosphorylated starch derivative, carbohydrate based rheological modifying agents, polymeric and copolymeric rheological modifying agents, inorganic rheological modifying agents, protein Theological modifying agents, polypeptide Theological modifying agents, and any combination of any of the foregoing.

The term “phosphorylated starch derivative” includes, but is not limited to, starches containing a phosphate group. Suitable phosphorylated starch derivatives include, but are not limited to, hydroxyalkyl starch phosphates, hydroxyalkyl distarch phosphates, and any combination of any of the foregoing. Non-limiting examples of hydroxyalkyl starch phosphates and hydroxyalkyl distarch phosphates include hydroxyethyl starch phosphate, hydroxypropyl starch phosphate, hydroxypropyl distarch phosphate (including sodium hydroxypropyl starch phosphate), and any combination of any of the foregoing.

Non-limiting examples of suitable carbohydrate based Theological modifying agents include algin and derivatives and salts thereof, such as algin, calcium alginate, propylene glycol alginate, and ammonium alginate; carrageenan (Chondrus crispus) and derivatives and salts thereof, such as calcium carrageenan and sodium carrageenan; agar; cellulose and derivatives thereof, such as carboxymethyl hydroxyethylcellulose, cellulose gum, cetyl hydroxyethylcellulose, hydroxyethylcellulose, hydroxypropylcellulose, hydroxypropyl methylcellulose, methylcellulose, ethylcellulose, and cellulose gum; chitosan and derivatives and salts thereof, such as hydroxypropyl chitosan, carboxymethyl chitosan, and chitin; gellan gum; guar (Cyanopsis tetragonoloba) and derivatives thereof, such as guar hydroxypropyltrimonium chloride and hydroxypropyl guar; hyaluronic acid and derivatives thereof, such as sodium hyaluronate; dextran and derivatives thereof; dextrin; locust bean (Ceratonia siliqua) gum; starches, such as starch polyacrylonitrile copolymer-potassium salt and starch polyacrylonitrile copolymer-sodium salt; pectin; sclerotium gum; tragacanth (Astragalus gumnifer) gum; xantham gum and derivatives thereof; and any combination of any of the foregoing.

Non-limiting examples of suitable polymeric and copolymeric rheological modifying agents include acrylates, methacrylates, polyethylene and derivatives thereof, and any combination of any of the foregoing. Suitable acrylates and methacrylates include, but are not limited to, carbomer and derivatives and salts thereof, acrylate/C₁₀-C₃₀ alkyl acrylate crosspolymer, acrylate/ceteth-20 itaconate copolymer, acrylate/ceteth-20 methacrylate copolymers, acrylate/steareth-20 methacrylate copolymers, acrylate/steareth-20 itaconate copolymers, acrylate/steareth-50 acrylate copolymers, acrylate/VA crosspolymers, acrylate/vinyl isodecanoate crosspolymers, acrylic acid/acrylonitrogen copolymers, ammonium acrylate/acrylonitrogen copolymers, glyceryl polymethacrylate, polyacrylic acid, PVM/MA decadiene crosspolymer, sodium acrylate/vinyl isodecanoate crosspolymers, sodium carbomer, ethylene/acrylic acid copolymer, ethylene/VA copolymer, acrylate/acrylamide copolymer, acrylate copolymers, acrylate/hydroxyester acrylate copolymers, acrylate/octylarylamide copolymers, acrylate/PVP copolymers, AMP/acrylate copolymers, butylester of PVM-MA copolymer, carboxylate vinylacetate terpolymers, diglycoVCHDM/isophthalates/SIP copolymer, ethyl ester of PVM-MA copolymer, isopropyl ester of PVM-MA copolymer, octylacrylamide/acrylate/butylaminoethyl methacrylate copolymers, polymethacrylamidopropyltrimonium chloride, propylene glycol oligosuccinate, polyvinylcaprolactam, PVP, PVP/dimethylaminoethylmethacrylate copolymer, PVP/DMAPA acrylate copolymers, PVP/carbamyl polyglycol ester, PVPNA copolymer, PVP/VA vinyl propionate copolymer, PVP/vinylcaprolactam/DMAPA acrylate copolymers, sodium polyacrylate, VA/butyl maleate/isobomyl acrylate copolymers, VZ/crotonates copolymer, VA/crotonates vinyl neodecanoate copolymer, VA crotonates/vinyl propionate copolymer, vinyl caprolactam/PVP/dimethylaminoethylmethacrylate copolymer, and any combination of any of the foregoing.

Non-limiting examples of suitable inorganic thickening agents include clays and derivatives thereof, silicates, silicas and derivatives thereof, and any combination of any of the foregoing. Suitable clays and derivatives thereof include, but are not limited to, bentonite and derivatives thereof, such as quaternium-18 bentonite; hectorite and derivatives thereof, such as quaternium-18 dectorite; montmorillonite; and any combination of any of the foregoing. Suitable silicates include, but are not limited to, magnesium aluminum silicate, sodium magnesium silicate, lithium magnesium silicate, tromethamine magnesium aluminum silicate, and any combination of any of the foregoing. Suitable silicas and derivatives thereof include, but are not limited to, hydrated silica, hydrophobic silica, and any combination of any of the foregoing.

Suitable protein and polypeptide rheological modifying agents include, but are not limited to, proteins and derivatives and salts thereof, polypeptides and derivatives and salts thereof, and any combination of any of the foregoing. Non-limiting examples of protein and polypeptide rheological modifying agents include albumin, gelatin, keratin and derivatives thereof, fish protein and derivatives thereof, milk protein and derivatives thereof, wheat protein and derivatives thereof, soy protein and derivatives thereof, elastin and derivatives thereof, silk protein and derivatives thereof, and any combination of any of the foregoing.

Preferred rheological modifying agents include, but are not limited to, carbomer, acrylate/alkyl acrylate crosspolymers, acrylate/vinyl isododecanoate crosspolymer, xantham gum, locust bean gum, guar gum, and any combination of any of the foregoing. A more preferred combination of rheological modifying agents comprises carbomer and an acrylate/alkyl acrylate copolymer, such as an acrylate/C10-C₃₀ alkyl acrylate crosspolymer. According to the International Cosmetic Ingredient Dictionary and Handbook (7^{th} Ed., The Cosmetic, Toiletry, and Fragrance Association), carbomer is a homopolymer of acrylic acid crosslinked with an allyl ether of pentaerythritol, an allyl ether of sucrose, or an allyl ether of propylene. The term “acrylate/alkyl acrylate crosspolymer” includes, but is not limited to, copolymers of alkyl acrylates with one or more monomers of acrylic acid, methacrylic acid, or one of their short chain (i.e. C.₁₋₄ alcohol) esters, wherein the crosslinking agent is, for example, an allyl ether of sucrose or pentaerytritol. Preferably, the alkyl acrylates are C₁₀-C₃₀ alkyl acrylates. Examples of such copolymers include, but are not limited to, those commercially available as Carbopol™ 1342, Carbopol™ 1382, Pemulen™ TR-1, and Pemulen™ TR-2, from Goodrich Specialty Chemicals of Cleveland, Ohio.

Preferred rheological modifying agents include, but are not limited to hydrophilic gelling agents, such as carboxyvinyl polymers (carbomer), acrylic copolymers (e.g. acrylate/alkyl acrylate copolymers), polyacrylamides, polysaccharides (e.g. hydroxypropylcellulose), natural gums, clays, and any combination of any of the foregoing.

The base composition typically comprises from about 0.001 to about 50% and preferably from about 0.01 to about 10%, and more preferably from about 0.1 to about 5% by weight of hydrophilic rheological modifying agents. The base composition typically comprises from about 0.001 to about 99.99%, preferably from about 1 to about 99.99%, and more preferably from about 20 to about 99.99% by weight of water.

Phospholipids which may comprise from 0.01% to 8% by weight (preferably 0.01 to 5% by weight) of the dispersion may include Phospholipon 80, 80H (American Lecithin Co., Oxford, Conn.), Basis LP20H (Ikeda Corp., Japan), and Catemol, a synthetic lipid-like compound (Phoenix Chemicals Inc., Somerville, N.J.).

The compositions of the present invention may also contain other physiologically active ingredients. Suitable active agents for the compositions prepared by the method of the invention include, but are not limited to, anti-acne agents, antimicrobial agents, anti-inflammatory agents, analgesics, antierythemal agents, antipruritic agents, antiedemal agents, antipsoriatic agents, antifungal agents, skin protectants, sunscreen agents, vitamins, antioxidants, scavengers, antiirritants, antibacterial agents, antiviral agents, antiaging agents, protoprotection agents, hair growth enhancers, hair growth inhibitors, hair removal agents, antidandruff agents, anti-seborrheic agents, exfoliating agents, wound healing agents, anti-ectoparacitic agents, sebum modulators, immunomodulators, hormones, botanicals, moisturizers, astringents, cleansers, sensates, antibiotics, anesthetics, steroids, tissue healing substances, tissue regenerating substances, amino acids, peptides, minerals, ceramides, biohyaluronic acids, enzymes and any combination of any of the foregoing.

Preferred anti-acne agents include, but are not limited to, salicylic acid, retinoic acid, alpha hydroxy acid, benzyl peroxide, sodium sulfacetamide, clindamycin, and any combination of any of the foregoing. Preferred combinations of anti-acne agents to be incorporated in the composition include salicylic acid, retinoic acid, and hydrocortisone; sodium sulfacetamide and clindamycin; salicylic acid and clindamycin; salicylic acid, alpha hydroxy acid, and tetrahydrozoline.

Suitable antimicrobial agents include, but are not limited to, benzalkonium chloride, benzethonium chloride, chlorhexidine gluconate, chloroxylenol, cloflucarban, fluorosalan, hexachlorophene, hexylresorcinol, iodine complex, iodine tincture, para-chloromercuriphenol, phenylmercuric nitrate, thimerosal, vitromersol, zyloxin, triclocarban, triclosan, methyl-benzethonium chloride, nonyl phenoxypoly(ethyleneoxy) ethanol-iodine, para-chloro-meta-xylenol, providone-iodine complex, poloxamer-iodine complex, triclorcarban, undecoylium chloride-iodine complex, and any combination of any of the foregoing.

Suitable anti-inflammatory agents include, but are not limited to, alidoxa, allantoin, aloe vera, aluminum acetate, aluminum hydroxide, bismuth subnitrate, boric acid, calamine, casein, cellulose, microporous, cholecatciferol, cocoa butter, cod liver oil, colloidal oatmeal, cystein hydrochloride, dexpanthenol, dimethicone, glycerin, kaolin, lanolin, live yeast cell derivative, mineral oil, peruvian balsam, petrolatum, protein hydrolysate, racemethionine, shark liver oil, sodium bicarbonate, sulfur, talc, tannic acid, topical starch, vitamin A, vitamin E, white petrolatum, zinc acetate, zinc carbonate, zinc oxide, hydrocortisone, betamethasone, ibuprofen, indomethicin, acetyl salicylic acid, tacrolimus, flucoinolone acetonide, sodium sulfacetamide, and any combination of any of the foregoing.

Suitable analgesics include, but are not limited to, diphenhydramine, tripeiennamine, benzocaine, dibucaine, lidocaine, tetracaine, camphor, menthol, phenol, resorcinol, matacresol, juniper tar, methylsalicylate, turpentine oil, capsicum, methyl nicotinate, b-glucan, and any combination of any of the foregoing.

Suitable antietythermal agents include, but is not limited to, tetrahydrozoline and hydracortisone.

Suitable antipruritic agents include, but are not limited to, diphenhydramine, pramoxine, antihistamines, and any combination of any of the foregoing.

Suitable antiedemal agents, include, but are not limited to, pregnenalone acetate, tannin glycosides, and any combination of any of the foregoing.

Suitable antipsoriatic agents include, but are not limited to, caleipotriene, coal tar, anthralin, vitamin A, and any combination of any of the foregoing. Preferred combinations of antipsoriatic agents include, but are not limited to, hydrocortisone, retinoic acid, and alpha hydroxy acid; dovonex, salicylic acid, and a sunscreen agent; indomethacin, salicylic acid, and urea; anthralin and salicylic acid; and anthralin and indomethacin. Other suitable antipsoriatic agents include, but are not limited to, caleipotriene, coal tar, anthralin, vitamin A, and any combination of any of the foregoing.

Suitable antifungal agents include, but are not limited to, clioquinol, haloprogin, miconazole nitrate, clotrimazole, metronidazole, toinaftate, undecylenic acid, iodoquinol, and any combination of any of the foregoing.

Suitable skin protectants include, but are not limited to, cocoa butter, dimethicone, petrolatum, white petrolatum, glycerin, lipidure, shark liver oil, allantoin, and any combination of any of the foregoing.

Suitable sunscreen agents include, but are not limited to, ethylhexyl methoxycinnamate, avobenzone, benzophenone-3, octacrylene, titanium dioxide, zinc oxide, and any combination of any of the foregoing.

Suitable antioxidants include, but are not limited to, scavengers for lipid free radicals and peroxyl radicals, quenching agents, and any combination of any of the foregoing. Suitable antioxidants include, but are not limited to, tocopherol, BHT, beta carotene, vitamin A, ascorbic acid, ubiquinol, ferulic acid, azelaic acid, thymol, catechin, sinapic acid, EDTA, lactoferrin, rosmariquinone, hydroxytyrosole, sesamol, 2-thioxanthine, nausin, malvin, carvacone, chalcones, glutathione isopropyl ester, xanthine, melanin, guanisone, lophorphyrins, 8-hydroxyxanthine, 2-thioxanthione, vitamin B₁₂, plant alkaloids, catalase, quercetin, tyrosine, SOD, cysteine, methionine, methylsulphonylmethane (SM), genistein, NDG, procyanidin, hamamelitannin, ubiquinone, trolox, licorice extract, propyl gallate, sinapic acid, and any combination of any of the foregoing.

Suitable vitamins include, but are not limited to, vitamin E, vitamin A palmitate, vitamin D, vitamin F, vitamin B₆, vitamin B₃, vitamin B₁₂, vitamin C, ascorbyl palmitate, vitamin E acetate, biotin, niacin, DL-panthenol, and any combination of any of the foregoing.

A preferred sunscreen agent is a mixture of ethylhexyl methoxycinnamate, butyl methoxydibenzoylmethane, cyclomethicone, phospholipids, and water, and is available as Solarease™ from Collaborative Laboratories, Inc. of East Setauket, N.Y.

Suitable amino acids include, but are not limited to, glycine, serine, and any combination of any of the foregoing.

In certain embodiments, compositions of the invention may comprise a preservative such as chlorophenesin, sorbic acid, disodium ethylenedinitrilotetraacetate, phenoxyethanol, methylparaben, ethylparaben, propylparaben, phytic acid, imidazolidinyl urea, sodium dehydroacetate, benzyl alcohol and benzoic acid. A preferred preservative is Phenonip (NIPA, Wilmington, Del.); Germazide™ MPB, available from Collaborative Laboratories, Inc. of East Setauket, N.Y.; and polyoxethylene ethers. The dispersion may also comprise polyethylene glycol (for example PEG 4 or PEG 8) and/or butylene glycol (for example, 1,3-butylene glycol) to improve the freeze thaw stability of the preparations.

Aesthetic Modifying Agents

The composition of the current invention may also contain other ingredients to enhance the aesthetic properties of the final preparation. An aesthetic modifying agent is a material that imparts desirable tactile, olfactory, taste or visual properties to the surface to which the composition is applied. The aesthetic modifying agent may be hydrophobic or hydrophilic. The aesthetic modifying agent is preferably hydrophobic and is more preferably an oil, wax, solid or paste.

A dispersion of one or more hydrophobic aesthetic modifying agents is preferably prepared before the hydrophobic aesthetic modifying agents are incorporated into the composition. The hydrophobic aesthetic modifying agents may be dispersed into an aqueous phase by methods known in the art, such as by ultra high shear mixing and microfluidization.

The final composition may be prepared by mixing the dispersions containing the hydrophobic aesthetic modifying agents with the base composition and any other adjuvants. Since the hydrophobic aesthetic modifying agents are added to the base composition as dispersions, heating and other expensive processing steps are not required to obtain a homogenous final composition.

An example of an aesthetic modifying agent is a mono, di, tri or poly alkyl ester or ether of a di, tri, or polyhydroxy compound, such as ethylene glycol, propylene glycol, glycerin, sorbitol or other polyol compound.

An example of a hydrophobic aesthetic modifying agent is a compound having the formula CₙH₍₂ₙ₊₂₋ₘ₎ wherein n is an integer greater than or equal to 6 and m is 0 or an even integer no greater than n. Such compounds include, but are not limited to, saturated and unsaturated, linear, branched, and cyclic hydrocarbon chains. Preferred examples of such compounds include, but are not limited to mineral oil, petrolatum, permethyl fluids, polybutylenes, and polyisobutylenes.

Another example of a hydrophobic aesthetic modifying agent has the formula

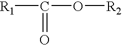

or the formula

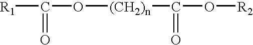

wherein R₁ is a saturated or unsaturated, linear, branched or cyclic C₁-C₂₄ alkyl group; R₂ is hydrogen or a saturated or unsaturated, liner, branched or cyclic C₁-C₂₄ alkyl group; and n is an integer from 0 to 20. Examples of such aesthetic modifying agents include, but are not limited to, isopropyl palmitate and diisopropyl adipate. Examples of such esters and ethers include but are not limited to, saturated and unsaturated, linear and branched vegetable oils, such as soybean oil, babassu oil, castor oil, cottonseed oil, chinese tallow oil, crambe oil, perilla oil, danish rapeseed oil, rice bran oil, palm oil, palm kernel oil, olive oil, linseed oil, coconut oil, sunflower oil, safflower oil, peanut oil and corn oil. Preferred saturated and unsaturated vegetable oils are those having fatty acid components with 6 to 24 carbon atoms. A more preferred vegetable oil is soybean oil.

Yet another aesthetic modifying agents are based on silicone chemistry. Silicone may provide lubrication and/or shine to the composition. Preferably, the silicone is insoluble in water. Suitable water-insoluble silicone materials include, but are not limited to, polyalkylsiloxanes, polyarylsiloxanes, polyalkylarylsiloxanes, polysiloxane gums and polyethersiloxane copolymers. Examples of suitable silicone materials are disclosed in U.S. Pat. Nos. 4,788,006; 4,341,799; 4,152,416; 3,964,500; 3,208,911; 4,364,837 and 4,465,619, all of which are incorporated herein by reference.

Another suitable hydrophobic material which can be suspended in the composition has the formula

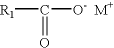

wherein R₁ is a saturated or unsaturated, linear, branched or cyclic alkyl having 2 to 24 carbon atoms; M⁽⁺⁾ is N⁺R₂R₃R₄R₅; where R₂, R₃ and R₄ are hydrogen or a saturated or unsaturated, linear or branched alkyl or hydroxyalkyl having from 1 to 10 carbon atoms; and R₅ is a saturated or unsaturated, linear, branched or cyclic alkyl or substituted alkyl having 2 to 24 carbon atoms. An example of such a material is lauramine oleate.

In order to illustrate farther the present invention, the experiments described in the following examples were conducted. It should be understood that the invention is not limited to the specific examples or the details described therein.

### EXAMPLES

It should be understood that the invention is not limited to the specific examples or the details described herein.

### Example 1

| | Ingredient | wt % |
|---|---|---|
| A | Deionized Water (0.2 μm filtered) | 45.80 |
| | Petrolatum Protopet White 1S¹ | 30.00 |
| | 200/100 Fluid² | 20.00 |
| | Vitamin E Acetate³ | 0.20 |
| | Germazide MPB⁴ | 1.50 |
| B | Catemol S-180S⁵ | 2.50 |

¹Witco, Greenwich, CN
²Dow Corning, Midland, MI
³Roche, Nutley, NJ
⁴The Collaborative Group, Ltd., Stony Brook, NY
⁵Phoenix Chemical, Somerville, NJ

1) Mixed Phase A at about 80 C.

2) Slowly added Phase B and mixed until uniform.

3) Subjected Phase AB to high pressure/high shear mixing with from 9,000 to 25,000 psi until a uniform preparation with the desired particle size was obtained. The pH of the dispersion was between 5.00 and 7.00. The specific gravity was between 0.92-0.95. The particle size was below 500 nm.

### Example 2

| | Ingredient | wt % |
|---|---|---|
| A | Deionized Water (0.2 μm filtered) | 45.70 |
| | Petrolatum Protopet White 1S¹ | 50.00 |
| | Germazide MPB² | 1.50 |
| B | Catemol S-180S³ | 2.75 |

¹Witco, Greenwich, CN
²The Collaborative Group, Ltd., Stony Brook, NY
³Phoenix Chemical, Somerville, NJ

1) Mixed Phase A at about 80 C.

2) Slowly added Phase B and mixed until uniform.

3) Subjected Phase AB to high pressure/high shear mixing with from 9,000 to 25,000 psi until a uniform preparation with the desired particle size was obtained.

The specific gravity of the dispersion was between 0.85-0.92. The pH of the dispersion was between 5.10 and 7.10. The particle size of the dispersion was below 650 nm. The viscosity was between 4000 and 9000 cps.

### Example 3

| | Ingredient | wt % |
|---|---|---|
| A | Deionized Water (0.2 μm filtered) | 43.80 |
| | Petrolatum Protopet White 1S¹ | 30.00 |
| | 200/100 Fluid² | 20.00 |
| | Fomblin HC-25⁶ | 2.00 |
| | Vitamin E Acetate³ | 0.20 |
| | Germazide MPB⁴ | 0.20 |
| B | Catemol S-180S⁵ | 2.50 |

¹Witco, Greenwich, CN
²Dow Corning, Midland, MI
³Roche, Nutley, NJ
⁴The Collaborative Group, Ltd., Stony Brook, NY
⁵Phoenix Chemical, Somerville, NJ
⁶Brooks, South Plainfield, NJ

1) Mixed Phase A at about 80 C.

2) Slowly added Phase B and mixed until uniform.

3) Subjected Phase AB to high pressure/high shear mixing with from 9,000 to 25,000 psi until a uniform preparation with the desired particle size was obtained.

The pH of the dispersion was between 5.00 and 7.00. The specific gravity was between 0.92-0.95. The particle size was below 500 nm.

### Example 4

| | Ingredient | wt % |
|---|---|---|
| A | Deionized Water (0.2 μm filtered) | 62.00 |
| | Phenonip¹ | 1.00 |
| B | Basis LP-20H² | 2.00 |
| C | Silicone 1401 Fluid³ | 10.00 |
| D | Petrolatum Protopet White 1S⁴ | 25.00 |

¹NIPA, Wilmington, DE
²Ikeda, Tokyo, Japan
³Dow Corning, Midland, MI
⁴Witco, Greenwich, CN

1) Heated Phases C & D to approximately 80° C. and mixed with prop until uniform.

2) Homogenized Phase A.

3) Slowly added Phase B and mixed until uniform.

4) Slowly added Phase CD and mixed until uniform.

5) Subjected Phase ABCD to high pressure/high shear mixing with from 9,000 to 25,000 psi until a uniform preparation with the desired particle size was obtained.

The pH of the dispersion was between 5.00 and 8.00. The specific gravity was between 0.92-0.98. The particle size was below 500 nm.

### Example 5

| | Ingredient | wt % |
|---|---|---|
| A | Deionized Water (0.2 μm filtered) | 82.75 |
| | Phenonip¹ | 1.00 |
| B | NAT 8729² | 6.00 |
| C | Silicone 200 Fluid/60,000 cst.³ | 10.00 |
| D | Keltrol CG-RD⁴ | 0.25 |

¹NIPA, Wilmington, DE
²American Lecithin, Oxford CT
³Dow Corning, Midland, MI
⁴Kelco, San Diego, CA

1) Homogenized Phase A.

2) Slowly added Phase B and mixed until uniform.

3) Slowly added Phase C and mixed until uniform.

4) Subjected Phase ABC to high pressure/high shear mixing with from 9,000 to 25,000 psi until a uniform preparation with the desired particle size was obtained.

5) Slowly added Phase D while mixing with prop until completely wet out and uniform.

The pH of the dispersion was between 5.00 and 8.00. The specific gravity was between 0.9500-1.050. The particle size was below 500 nm.

### Example 6

| | Ingredient | wt % |
|---|---|---|
| A | Deionized Water (0.2 μm filtered) | 44.8499 |
| A | White Protopet 1S¹ | 50.00 |
| B | Burst RSD-10² | 0.05 |
| B | Lipidure HM 4000S³ | 0.0001 |
| B | Phenonip⁴ | 1.00 |
| B | Basis LP-20H⁵ | 4.00 |
| C | Keltrol CG-RD⁶ | 0.10 |

¹Witco, Greenwich, CN
²Hydrolabs, Albemarle, NC
³Oleo, Stony Brook, NY
⁴NIPA, Wilmington, DE
⁵Ikeda, Tokyo, Japan
⁶Kelco, San Diego, CA

1) Heated Phases A & B to approximately 80° C. and mixed with prop until uniform

2) Homogenized Phase A & B until uniform.

3) Subjected Phase AB to high pressure/high shear mixing with from 9,000 to 25,000 psi until a uniform preparation with the desired particle size was obtained.

4) Mixed with prop while slowly adding Phase C until completely dispersed (Wet out) and uniform.

The pH of the dispersion was between 5.50 and 7.75. The specific gravity was between 0.85-0.94. The particle size was below 500 nm.

### Example 7

| | Ingredient | wt % |
|---|---|---|
| A | Deionized Water (0.2 μm filtered) | 61.4999 |
| A | Silicone Fluid 345¹ | 30.00 |
| B | PEG-8² | 5.00 |
| B | Lipidure EM 4000S³ | 0.0001 |
| B | Phenonip⁴ | 1.20 |
| B | Basis LP-20H⁵ | 2.00 |
| C | Phospholipon 80H⁶ | 0.30 |

¹Dow Corning, Midland, MI
²Pride, Holtsville, NY
³Oleo, Stony Brook, NY
⁴NIPA, Wilmington, DE
⁵Ikeda, Tokyo, Japan
⁶American Lecithin, Oxford CT

1) Homogenized Phase A, B & C until uniform.

2) Subjected Phase ABC to high pressure/high shear mixing with from 9,000 to 25,000 psi until a uniform preparation with the desired particle size was obtained.

The pH of the dispersion was between 5.50 and 7.75. The specific gravity was between 0.95-1.10. The particle size was below 500 nm.

### Example 8

| | Ingredient | wt % |
|---|---|---|
| A | Deionized Water (0.2 μm filtered) | 62.4999 |
| A | Lipidure HM 4000S¹ | 0.0001 |
| B | Phenonip² | 1.00 |
| B | Basis LP-20H³ | 1.50 |
| C | Silicone Fluid 556⁴ | 20.00 |
| C | Silicone Fluid 1401⁴ | 10.00 |
| D | Deionized Water (0.2 μm filtered) | 4.7975 |
| D | Phenonip² | 0.05 |
| E | Carbomer 934⁵ | 0.10 |
| F | Triethanolamine⁶ | 0.0525 |

¹Oleo, Stony Brook, NY
²NIPA, Wilmington, DE
³Ikeda, Tokyo, Japan
⁴Dow Corning, Midland, MI
⁵BF-Goodrich, Cleveland, OH
⁶KCl (Kramer-Jopack) Glen Rock, NJ

1) Homogenized Phases A, B & C until uniform.

2) Subjected Phase ABC to high pressure/high shear mixing with from 9,000 to 25,000 psi until a uniform preparation with the desired particle size was obtained.

3) Separately mixed Phase D with prop until uniform.

4) Added Phase E to Phase D and mixed until uniform.

5) Add Phase F to Phase DE and mixed until uniform.

6) Added Phase DEF to Microfluidized product and mixed until uniform.

The pH of the dispersion was between 4.50 and 7.00. The specific gravity was between 0.9500-1.1000. The particle size was below 500 nm.

### Example 9

### Moisturizer Efficacy Test on Legs for Skin Care Formulations and/or Ingredients

This study was performed to determine the clinical effects of compositions of the invention

Materials and Methods

This was a randomized, double-blind study. A total of 20 female subjects (27-55 y.o.) completed the study. Subjects underwent a seven-day home conditioning phase and a one-day product application phase.

Test Samples

Composition of Example 1

20% Silicone in cationic vehicle

Composition of Example 2

Composition of Example 6

White Protapet IS Petrolatum

Conditioning Phase

Subjects providing informed consent and meeting inclusion/exclusion criteria underwent a seven-day home conditioning phase during which they discontinued moisturizer use and all skin care products on their lower legs and used Neutrogena Original cleansing bar for all general cleansing. Following the conditioning period, the lower outer legs of each subject were evaluated.

Those found to have satisfactory dryness continued on to the one-day product application phase.

Product Application Phase

The lateral aspect of each leg was divided into four test sites (3×4 cm² each.) A single application of 25 ul of assigned test product was applied to each test site. Using positive displacement pipettes, study personnel dispensed product directly onto each test site and rubbed it into the skin for approximately 10 seconds using a gloved finger. One test site per subject served as an untreated control.

Bioinstrumentation

Skin barrier integrity of the lower outer legs was monitored over six hours using a DermaLab with Water Loss Module to measure TEWL. TEWL measurements were taken at baseline and six hours after product application. Paired comparisons on the change from baseline TEWL for each test product were made using a General Linear Model ANOVA. An F test was conducted to determine overall statistical significance at p≦0.10. If overall statistical differences were detected, paired comparisons were made among the test products to determine statistical significance at p≦0.05.

Results

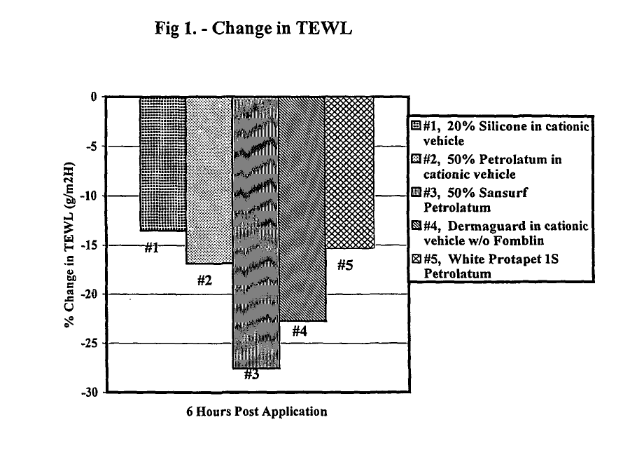

FIG. 1 shows the change in TEWL. Sites treated with 20% Silicone in cationic vehicle improved TEWL by 13.5 percent. Sites treated with Example 2 improved TEWL by 17 percent. Sites treated with Example 6 improved TEWL by 28 percent. Sites treated with Example 1 improved TEWL by 23 percent. Sites treated with White Protapet IS Petrolatum improved TEWL by 15 percent.

### CONCLUSION

When applied to the skin, the compositions of the invention reduced the loss of water vapor from the skin compared to petrolatum alone thereby creating an enhanced barrier film.

All patents, publications, applications, and test methods mentioned herein are hereby incorporated by reference. Many variations of the present invention will suggest themselves to those skilled in the art in light of the above, detailed description. All such obvious variations are within the full intended scope of the appended claims.

What is claimed is:

1. A method for preparing a topical composition with enhanced barrier properties comprising mixing:
(a) an oil and water dispersion comprising one or more skin protective agents selected from the group consisting of petrolatum, silicone, or a fluorocarbon, wherein said dispersion has a particle size of from about 50 to 1000 nm; and
(b) a base composition comprising
(i) a hydrophilic rheological modifying agent; and
(ii) an aqueous phase.

2. The method of claim 1 wherein the base composition comprises from about 0.01 to about 10% by weight of the hydrophilic Theological modifying agent.

3. The method of claim 1, wherein the hydrophilic rheological modifying agent is selected from the group consisting of phosphorylated starch derivatives, carbohydrate based Theological modifying agents, polymeric and copolymeric rheological modifying agents, inorganic Theological modifying agents, protein Theological modifying agents, polypeptide Theological modifying agents, and any combination of any of the foregoing.

4. The method of claim 1 wherein the hydrophilic Theological modifying agent is selected from the group consisting of carbomer, acrylate/alkyl acrylate crosspolymers, acrylate/vinyl isododecanoate crosspolymer, xantham gum, locust bean gum, and guar gum or a mixture of any of the foregoing.

5. The method of claim 1 wherein the hydrophilic rheological modifying agent comprises carbomer or an acrylate/alkyl acrylate crosspolymer.

6. The method of claim 5 wherein the acrylate/alkyl acrylate crosspolymer is acrylate/C₁₀-C₃₀ alkyl acrylate crosspolymer.

7. The method of claim 1, wherein the dispersion is produced using high pressure processing, high shear processing, or a combination thereof.

8. The method of claim 1, wherein the fluorocarbon is selected from the group consisting of linear, saturated, unsaturated, cyclic or branched compounds containing one or more mono-, di-, or tri-fluoro substituted methylene units or a mixture of any of the foregoing.

9. The method of claim 1 wherein the fluorocarbon is perfluoropolymethylisopropyl ether.

10. The method of claim 1 further comprising a preservative which is selected from one or more members of the group consisting of chlorophenesin, sorbic acid, disodium ethylenedinitrilotetraacetate, phenoxyethanol, methylparaben, ethylparaben, propylparaben, phytic acid, imidazolidinyl urea, sodium dehydroacetate, benzyl alcohol and benzoic acid.

11. The method of claim 1 further comprising a pH adjuster, an aesthetic modifier, a chelating agent, a colorant, a fragrance, an odor masking agent, or any combination thereof.

12. The method of claim 1, wherein the skin protective agent is present in an amount from 10 to 60 wt %; preferably from about 25 to 55 wt %, based on the total weight of the composition.

13. The method of claim 1, wherein the topical composition has a pH of from about 5.0 to 7.0.

14. The method of claim 1, wherein the topical composition has a specific gravity of about 0.80 to 1.1.

15. A topical composition having enhanced barrier properties prepared by the method of claim 1.

16. The composition of claim 15, wherein the base composition comprises from about 0.01 to about 10% by weight of the hydrophilic rheological modifying agent.

17. The composition of claim 15, wherein the hydrophilic Theological modifying agent is selected from the group consisting of phosphorylated starch derivatives, carbohydrate based Theological modifying agents, polymeric and copolymeric rheological modifying agents, inorganic rheological modifying agents, protein Theological modifying agents, polypeptide Theological modifying agents, and any combination of any of the foregoing.

18. The composition of claim 15 wherein the hydrophilic Theological modifying agent is selected from the group consisting of carbomer, acrylate/acrylate crosspolymers, acrylate/vinyl isododecanoate crosspolymer, xantham gum, locust bean gum, and guar gum or a mixture of any of the foregoing.

19. The composition of claim 15 wherein the hydrophilic rheological modifying agent comprises carbomer or an acrylate/alkyl acrylate crosspolymer.

20. The method of claim 10 wherein the acrylate/alkyl acrylate crosspolymer is acrylate/C₁₀-C₃₀ alkyl acrylate crosspolymer.

21. The composition of claim 15, wherein the dispersion is produced using high pressure processing, high shear processing, or a combination thereof.

22. The composition of claim 15, wherein the fluorocarbon is selected from the group consisting of linear, saturated, unsaturated, cyclic or branched compounds containing one or more mono-, di-, or tri-fluoro substituted methylene units or a mixture of any of the foregoing.

23. The composition of claim 15 wherein the fluorocarbon is perfluoropolymethylisopropyl ether.

24. The composition of claim 15 further comprising a preservative which is selected from one or more members of the group consisting of chlorophenesin, sorbic acid, disodiurn ethylenedinitrilotetraacetate, phenoxyethanol, methylparaben, ethylparaben, propylparaben, phytic acid, imidazolidinyl urea, sodium dehydroacetate, benzyl alcohol and benzoic acid.

25. The composition of claim 15 further comprising a pH adjuster, an emollient, a chelating agent, a colorant, a fragrance, an odor masking agent, or any combination thereof.

26. The composition of claim 15 for topical, anal, vaginal, otic, ophthalmic, oral or nasal application.
